(19) United States
(12) Patent Application Publication
(10) Pub. No.: US 2006/0200148 A1
(43) Pub. Date: Sep. 7, 2006

(54) Device for the fixation of bones

(76) Inventor: Romano Matthys, Fideris (CH)

Correspondence Address: JONES DAY, 222 EAST 41ST STREET, NEW YORK, NY, 10017-6702, US

(21) Appl. No.: 11/390,800

(22) Filed: Mar. 27, 2006

Related U.S. Application Data
(63) Continuation of application No. PCT/CH03/00697, filed on Oct. 24, 2003.

(51) Int. Cl. A61F 2/30

(52) U.S. Cl. 606/69

(57) ABSTRACT

The device for bone fixation comprises a bone plate (1), made from a polymeric material, with an upper side (2), an underside (3) intended to be in contact with the bone and plate holes (4) of diameter “D”, which connect the upper side (2) with the underside (3), and with one hole axis (5) and several longitudinal bone fixation elements (10), which are intended to be introduced into the holes (4) of the plate (1) and have an external thread (11) with a diameter “A” and a core diameter “K”.

### CROSS-REFERENCE TO RELATED APPLICATIONS

This application is a continuation of International Application No. PCT/CH2003/000697, filed Oct. 24, 2003, the entirety of which is incorporated by reference herein.

### FIELD OF THE INVENTION

The invention relates to a device for the fixation of bones.

### BACKGROUND OF THE INVENTION

Such devices are suitable particularly for the medical care of bone fractures in regions in the vicinity of a joint, for example, at the proximal humerus.

The WO 01/1281 discloses a generic device. However, the internal threads, provided optionally in the plate boreholes, are dimensioned identically with the external thread of the longitudinal bone-fixation agents, so that the latter cannot cut into the material of the bone plate and, as a result, neither an axial nor a rotative stability is achieved.

The WO 01/93768 discloses a bone plate, which consists of a bioabsorbable plastic. However, there are only depressions in the plate instead of boreholes through the plate. The disadvantage of this known plate consists accordingly therein that the longitudinal bone fixation agents, brought into the depressions, must be drilled completely through the remaining thickness of the plate. On the one hand, special longitudinal bone fixation agents with a drilled tip are required for this purpose and, on the other, due to this procedure, chips or shavings are formed in a larger amount, which remain in the body at least for a certain time.

### SUMMARY OF THE INVENTION

It is therefore an object of the invention to create a device for the fixation of bones, which is in a position to connect two or more longitudinal bone fixation elements angularly stably and rotationally secure with one another over a bone plate, consisting of a polymeric plastic.

The advantages, achieved by the invention, can be seen to lie essentially therein that the anchoring threads are formed during the implantation of the bone fixation elements in the bone plate, consisting of a polymeric plastic. During this transformation process, a harmless amount of abraded material but no actual chips are formed. Due to the transformation of the anchoring thread while the longitudinal bone fixation element is being screwed into the bone plate, this element is connected angularly stably and rotationally secure with the bone plate. This connecting technique gives the user a certain latitude with regard to the positional accuracy of the longitudinal bone fixation element with respect to the borehole in the bone plate.

The bone plate of the inventive device has plate holes, which have a diameter, which is smaller than the diameter of the external thread of the longitudinal bone fixation agents, which are to be introduced into the plate holes, yet larger than the core diameter of this external thread. Due to these diameter differences, a corresponding thread is formed in situ in the plate holes of the bone plate when the longitudinal bone fixation agent is screwed into an appropriate thread. The radial prestressing, acting at the external thread of the longitudinal bone fixation agent in the contact zone (plate hole) of the bone plate, resulting from the thread transformation, secure the longitudinal bone fixation agent adequately with respect to the rotational moments acting upon it. This, in turn, prevents longitudinal shifting of the longitudinal bone fixation agent relative to the bone plate.

In the case of a particularly preferred embodiment, the bone plate consists of an essentially not absorbable plastic, preferably of PEEK (Polyetheretherketone). By these means, a particularly good fixation of the bone plate is surprisingly achieved.

Additionally, the bone plate may comprise a central borehole for accommodating a targeting device.

In the case of a further embodiment, the axes of one or more of the N plate holes make an angle alpha of more than 0° with the normal to the bone plate.

The quantity “D” for the diameter of the plate holes preferably is at least 1.05 “K” and not more than 1.15 “K” (“K” being the core diameter of the bone fixation element).

The quantity “A” for the diameter of the external thread of the bone fixation elements preferably is at least 1.2 K and not more than 1.3 K.

For a further embodiment, the axes of at least two and preferably of at least three of the N plate holes extend skew to one another. By these means, the anchoring of the device as a whole in the bone is improved.

### BRIEF DESCRIPTION OF THE DRAWINGS

The invention and further developments of the invention are described in even greater detail in the following by means of partially diagrammatic representations of several examples. In the drawings,

FIG. 1 shows a perspective view of the inventive device,

FIG. 2 shows a plan view of the bone plate of FIG. 1,

FIG. 3 shows a longitudinal section along the line III-III in FIG. 2,

FIG. 4 shows a longitudinal section along the line IV-IV in FIG. 2, and

FIG. 5 shows an enlarged partial section from FIG. 3.

### DETAILED DESCRIPTION OF THE INVENTION

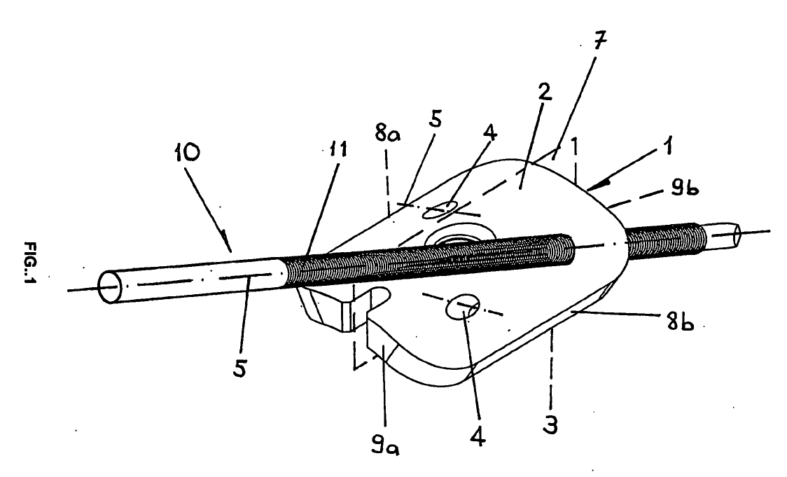

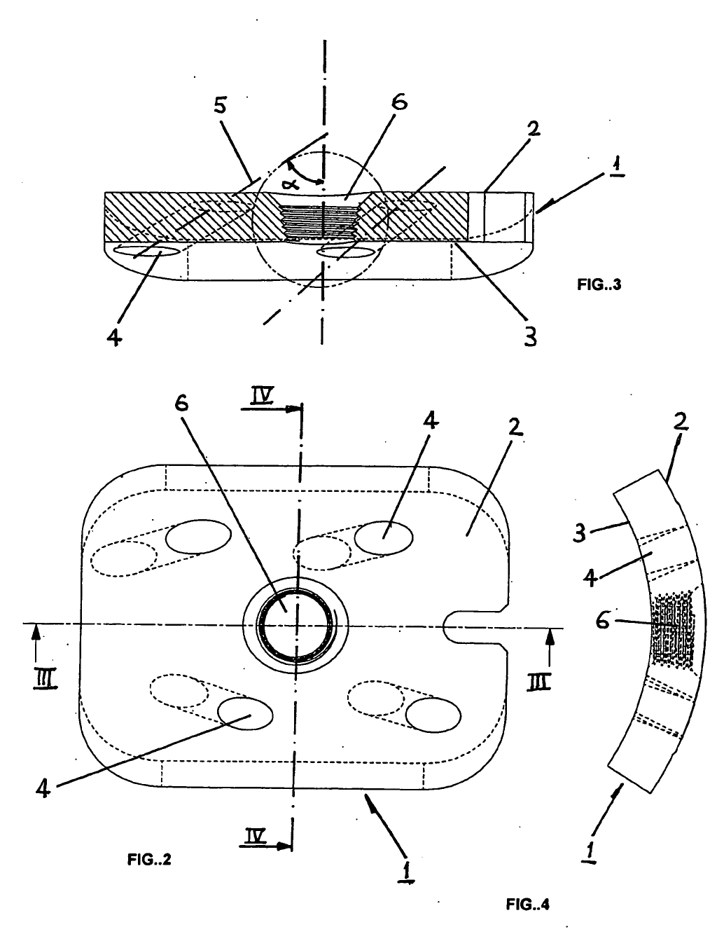

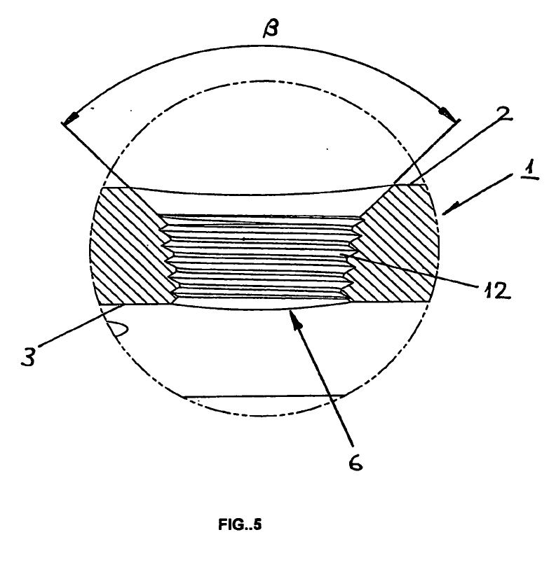

FIGS. 1 to 5 show an embodiment, which comprises a convexly curved bone plate 1 and a bone fixation element 10, which is screwed into one of the plate holes 4. The bone plate 1 is oval with rounded corners and, in cross-sectional areas orthogonal to the middle plane 7, is curved convexly. The axes 5 of the four plate holes 4 are disposed skew relative to one another and inclined to the upper side 2 of the bone plate 1. In the embodiment shown here, the bone plate 1 has two long side surfaces 8a, 8b and two short side surfaces 9a, 9b, as well as a middle plane 7 intersecting the two short side surface 9a, 9b. Only the body of the bone plate 1 is constructed symmetrically with respect to the middle plane 7. On the other hand, the plate holes 4 are not disposed symmetrically to the middle plane 7. The distances of the axes 5 of the plate holes 4 from the middle plane 7 as well as from the short side surfaces 9a, 9b of the bone plate 1 differ for the four plate holes 4. Furthermore, the axes 5 of the four plate holes 4 make an angle α of between 20° and 80° with the normal on the bone plate 1.

The plate holes 4 pass through the bone plate 1 in such a manner, that the intersections of the hole axes 5 with the upper side 2 are closer to the second short side surface 9b than to the underside 3. Accordingly, all the bone fixation elements 10 can be screwed into the bone plate 1 from the same side.

The plate holes 4 have a diameter, which is smaller than the external diameter of the external thread 11 of the bone fixation agent 10. Moreover, since the core diameter of the external thread 11 is smaller than the diameter of the plate holes 4, the bone fixation agents 10, on being screwed into the bone plate 1, are fixed to the latter by the transformation of the wall material in the borehole. The bone plate 1 is made from a polymeric material, so that the external threads 11 of the bone fixation elements 10 can be screwed into the bone plate 1 more easily.

For fastening a targeting device (not shown), the bone plate 1 comprises a central borehole 6, which passes through the bone plate 1 centrally from the upper side 2 up to the underside 3. The central borehole 6 is constructed conically and provided on the longitudinal section x adjoining the underside 3 with a conical internal thread 12. The thread-free longitudinal section y of the central borehole 6, adjoining the upper side 2, is constructed with a larger conical angle β. In the embodiment, shown here, x>y.

1. A bone fixation system comprising:
a bone plate having a upper surface, a lower surface for contacting bone, and at least a first fixation hole having a first diameter and an interior surface;
a first longitudinal element having a threaded portion having a second diameter, and configured to be inserted into the first fixation hole;
wherein the second diameter is larger than the first diameter, such that as the first longitudinal element is inserted into the first fixation hole, a thread is formed on the interior surface of the first fixation hole.

2. The system of claim 1, wherein the bone plate is comprised of a polymeric material.

3. The system of claim 2, wherein the polymeric material is PEEK.

4. The system of claim 1, further comprising a second fixation hole configured to receive a second longitudinal element.

5. The system of claim 1, further comprising a borehole having a central axis substantially perpendicular to at least one of the upper and lower surface of the bone plate.

6. The system of claim 5, wherein the borehole is at least partially threaded.

7. The system of claim 5, wherein the borehole is located in a substantially central location on the bone plate.

8. The system of claim 5, wherein the borehole is substantially conical.

9. The system of claim 1, wherein the first fixation hole has a first central axis forming a substantially angular relationship with a central axis of the bone plate.

10. The system of claim 9, wherein the angle created between the first central axis and the central axis of the bone plate is between about 20° and about 80°.

11. The system of claim 9, further comprising a second fixation hole having a second central axis substantially parallel to the first central axis.

12. The system of claim 9, further comprising a second fixation hole having a second central axis substantially askew to the first central axis.

13. The system of claim 1, wherein the lower surface of the bone plate is curved.

14. The system of claim 1, wherein the first longitudinal element has a core diameter, and wherein the first diameter is greater than the core diameter.

15. A method of bone fixation comprising:
(a) providing a bone plate having a upper surface, a lower surface for contacting bone, and at least a first fixation hole having a first diameter and an unthreaded interior surface;
(b) placing the bone plate proximate a bone;
(c) inserting a first longitudinal element into the first fixation hole, wherein the first longitudinal element has a threaded portion having a second diameter; and
(d) forming a thread is formed on the interior surface of the first fixation hole during insertion of the first longitudinal element therein.

16. The method of claim 15, wherein the bone plate is comprised of a polymeric material.

17. The method of claim 16, wherein the polymeric material is PEEK.

18. The method of claim 15, wherein the second diameter is larger than the first diameter.

19. The method of claim 15, wherein the bone plate further comprises a second fixation hole, and further comprising the step of inserting a second longitudinal element into the second fixation hole.
